(19) United States
(12) Patent Application Publication
(10) Pub. No.: US 2019/0233857 A1
(43) Pub. Date: Aug. 1, 2019

(54) METHODS AND MATERIALS FOR THE BIOSYNTHESIS OF COMPOUNDS INVOLVED IN SERINE METABOLISM AND DERIVATIVES AND COMPOUNDS RELATED THERETO

(71) Applicant: INVISTA NORTH AMERICA S.A.R.L., Wilmington, DE (US)

(72) Inventors: Daniel Bawdon, Redcar (GB); Mariusz Stanislaw Kamionka, Redcar (GB); Alexander Brett Foster, Redcar (GB); Arghya Barman, Redcar (GB)

(73) Assignee: INVISTA NORTH AMERICA S.A.R.L., Wilmington, DE (US)

(21) Appl. No.: 16/264,774

(22) Filed: Feb. 1, 2019

Related U.S. Application Data
(60) Provisional application No. 62/624,902, filed on Feb. 1, 2018.

(51) Int. Cl. C12P 13/06; C12N 15/74; C12N 9/04; C12N 9/16; C12N 9/10

(52) U.S. Cl. CPC C12P 13/06; C12N 15/74; C12N 9/0006; C12Y 206/01052; C12N 9/16; C12Y 301/03003; C12N 9/1096; C12Y 101/01095

(57) ABSTRACT

Biosynthetic methods and materials for the production of compounds involved in serine metabolism, derivatives thereof and/or compounds related thereto are provided. Also provided are products produced in accordance with these methods and materials.

This patent application claims the benefit of U.S. Provisional Application Ser. No. 62/624,902 filed Feb. 1, 2018, the contents of which are incorporated herein by reference in their entirety.

### FIELD

The present invention relates to biosynthetic methods and materials for the production of compounds involved in serine metabolism, derivatives thereof and compounds related thereto. The present invention also relates to products biosynthesized or otherwise encompassed by these methods and materials.

Replacement of traditional chemical production processes relying on, for example fossil fuels and/or potentially toxic chemicals, with environmentally friendly (e.g., green chemicals) and/or “cleantech” solutions is being considered, including work to identify building blocks suitable for use in the manufacturing of such chemicals. See, “Conservative evolution and industrial metabolism in Green Chemistry”, Green Chem., 2018, 20, 2171-2191.

L-serine is a widely used amino acid that has been proposed as a potential building block in biochemical processes.

Various attempts have been reported to produce large quantities of L-serine in Escherichia coli.

For example, in one reported approach, involving overexpressing the three L-serine synthetic genes at a suitable expression level, blocking the degradation of L-serine to pyruvate, and regulating the glyoxylate pathway, an L-serine producing E. coli strain was constructed (Gu et al. J Ind Microbiol Biotechnol 2014 41:1443-1450).

Toxicity of L-serine is a challenge for high-titer production in E. coli, Mundhada et al. (Metabolic Engineering 2017 39:141-150) disclosed an engineered E. coli lacking L-serine degradation pathways reportedly evolved for improved tolerance, by gradually increasing L-serine concentrations from 3 g/L to 100 g/L in an adaptive laboratory evolution (“ALE”) approach.

Li et al. (Biotechnol Lett (2012) 34:1525-1530) disclosed an engineered E. coli strain which accumulates L-serine, wherein the L-serine deaminase genes sdaA, sdaB and tdcG were deleted and serine was said to accumulate.

Biosynthetic materials and methods, including organisms having increased production of compounds involved in serine metabolism, and/or derivatives thereof and/or compounds related thereto, are needed.

### SUMMARY OF THE INVENTION

An aspect of the present invention relates to a process for biosynthesis of compounds involved in serine metabolism, including derivatives thereof and/or compounds related thereto. A process of the present invention comprises obtaining an organism capable of producing compounds involved in serine metabolism, and/or derivatives thereof and/or compounds related thereto, altering or engineering the organism, and producing more compounds involved in serine metabolism, and/or derivatives thereof and compounds related thereto in the altered organism as compared to the unaltered organism. In one nonlimiting embodiment, the organism is C. necator or an organism with properties similar thereto. In one nonlimiting embodiment, the organism is altered to overexpress one or more polypeptides which are capable of catalyzing the biosynthesis of L-serine from 3-phosphoglycerate. In one nonlimiting embodiment, the organism is altered to overexpress one or more enzymes capable of catalyzing the biosynthesis of L-serine from 3-phosphoglycerate.

In one nonlimiting embodiment, the organism is altered to overexpress a D-3-phosphoglycerate dehydrogenase or a polypeptide having the activity of a D-3-phosphoglycerate dehydrogenase. In one nonlimiting embodiment, the D-3-phosphoglycerate dehydrogenase is SerA. In one nonlimiting embodiment, the enzyme is SerA or SerA4 from Corynebacterium glutamicum or Cupriavidus necator, respectively. In one nonlimiting embodiment, the SerA comprises a polypeptide encoded by a nucleic acid sequence of SEQ ID NO: 1 or 6, a polypeptide with similar enzymatic activities and/or exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 1 or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 6 or a functional fragment thereof. In one nonlimiting embodiment, the D-3-phosphoglycerate dehydrogenase enzyme is classified in EC 1.1.1.95.

In one nonlimiting embodiment, the organism is altered to express a phosphoserine phosphatase or a polypeptide having the activity of a phosphoserine phosphatase. In one nonlimiting embodiment, the phosphoserine phosphatase is SerB. In one nonlimiting embodiment, the enzyme is SerB or SerB1 from E. coli or C. necator, respectively. In one nonlimiting embodiment, the SerB comprises a polypeptide encoded by a nucleic acid sequence of SEQ ID NO: 3, 4, 8 or 9, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 3, 4, 8 or 9 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 3, 4, 8 or 9 or a functional fragment thereof. In one nonlimiting embodiment, the phosphoserine phosphatase is classified in EC 3.1.3.3.

In one nonlimiting embodiment, the organism is altered to express a phosphoserine aminotransferase or a polypeptide having the activity of a phosphoserine aminotransferase. In one nonlimiting embodiment, the phosphoserine aminotransferase is SerC. In one nonlimiting embodiment, the SerC is from E. coli or C. necator. In one nonlimiting embodiment, the SerC comprises a polypeptide encoded by a nucleic acid sequence of SEQ ID NO: 2 or 7, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 2 or 7 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or 7 or a functional fragment thereof. In one nonlimiting embodiment, the phosphoserine aminotransferase is classified in EC 2.6.1.52.

In one nonlimiting embodiment, the nucleic acid sequence is codon optimized for C. necator.

In one nonlimiting embodiment, the organism is altered to express two or more of the enzymes of D-3-phosphoglycerate dehydrogenase, phosphoserine phosphatase and/or phosphoserine aminotransferase as disclosed herein.

In one nonlimiting embodiment, the organism is altered to express D-3-phosphoglycerate dehydrogenase, phosphoserine phosphatase and phosphoserine aminotransferase as disclosed herein.

In one nonlimiting embodiment, the organism is further altered to eliminate one or more genes encoding enzymes utilizing L-serine by other pathways. In one nonlimiting embodiment the eliminated one or more genes are sdaA and/or glyA.

In one nonlimiting embodiment, the organism is further altered to comprise a racemase to divert the carbon flux from L-Serine to D-Serine. In one nonlimiting embodiment, the racemase is a lysine/arginine racemase. In one nonlimiting embodiment, the lysine/arginine racemase is argR from Pseudomonas taetrolens. In one nonlimiting embodiment, the racemase comprises a polypeptide encoded by a nucleic acid sequence of SEQ ID NO: 5, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 5 or a functional fragment thereof, or a polypeptide with similar enzymatic activities encoded by a nucleic acid sequence with at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO: 5 or a functional fragment thereof. In one nonlimiting embodiment, the racemase is classified in EC 5.1.1.5, EC 5.1.1.9 or EC 5.1.1.18. In one nonlimiting embodiment, the organism is further modified to eliminate phaCAB, involved in PHBs production and/or H16-A0006-9 encoding endonucleases thereby improving transformation efficiency.

Another aspect of the present invention relates to an organism altered to produce more compounds involved in serine metabolism and/or derivatives and compounds related thereto as compared to the unaltered organism. In one nonlimiting embodiment, the organism is C. necator or an organism with properties similar thereto. In one nonlimiting embodiment, the organism is altered to overexpress one or more enzymes which catalyze the biosynthesis of L-serine from 3-phosphoglycerate. In one nonlimiting embodiment, the organism is altered to overexpress a D-3-phosphoglycerate dehydrogenase, a phosphoserine phosphatase and/or a phosphoserine aminotransferase as disclosed herein.

In one nonlimiting embodiment, the organism is altered with a nucleic acid sequence codon optimized for C. necator.

In one nonlimiting embodiment, the organism is further altered to eliminate one or more genes encoding enzymes utilizing L-serine by other pathways. In one nonlimiting embodiment the eliminated one or more genes are sdaA and/or glyA.

In one nonlimiting embodiment, the organism is further altered to comprise a racemase to divert the carbon flux from L-Serine to D-Serine. In one nonlimiting embodiment, the racemase is a lysine/arginine racemase as disclosed herein.

In one nonlimiting embodiment, the organism is further modified to eliminate phaCAB, involved in PHBs production and/or H16-A0006-9 encoding endonucleases thereby improving transformation efficiency.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof, as well as bio-derived, bio-based, or fermentation-derived C2 and C3 compounds with functional side chains comprising these bio-derived, bio-based, or fermentation-derived compositions or compounds; molded substances obtained by molding the bio-derived, bio-based, or fermentation-derived C2 and C3 compounds with functional side chains or the bio-derived, bio-based; bio-derived, bio-based, or fermentation-derived formulations comprising the bio-derived, bio-based, or fermentation-derived compositions or compounds, C2 and C3 compounds with functional side chains, or the bio-derived, bio-based, or fermentation-derived molded substances, or any combination thereof; and bio-derived, bio-based, or fermentation-derived semi-solids or non-semi-solid streams comprising the bio-derived, bio-based, or fermentation-derived compositions or compounds, C2 and C3 compounds with functional side chains, molded substances or formulations, or any combination thereof.

Another aspect of the present invention relates to a bio-derived, bio-based or fermentation derived product biosynthesized in accordance with the exemplary central metabolism depicted in FIG. 1.

Another aspect of the present invention relates to exogenous or non-exogenous genetic molecules of the altered organisms disclosed herein. In one nonlimiting embodiment, the exogenous genetic molecule comprises a codon optimized nucleic acid sequence encoding one or more enzymes which catalyze the biosynthesis of L-serine from 3-phosphoglycerate. In one nonlimiting embodiment, the exogenous genetic molecule comprises a codon optimized nucleic acid sequence encoding a D-3-phosphoglycerate dehydrogenase, a phosphoserine phosphatase and/or a phosphoserine aminotransferase as disclosed herein. In one nonlimiting embodiment, the nucleic acid sequence is codon optimized for C. necator. In one nonlimiting embodiment, the exogenous genetic molecule comprises a nucleic acid sequence encoding a D-3-phosphoglycerate dehydrogenase such as SerA. In one nonlimiting embodiment, the nucleic acid sequence comprises SEQ ID NO:1 or 6 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NOs: 1 or 6 or a functional fragment thereof. In one nonlimiting embodiment, the exogenous genetic molecule comprises a nucleic acid sequence encoding a phosphoserine phosphatase such as SerB. In one nonlimiting embodiment the nucleic acid sequence comprises SEQ ID NO:3, 4, 8 or 9 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:3, 4, 8 or 9 or a functional fragment thereof. In one nonlimiting embodiment, the exogenous genetic molecule comprises a nucleic acid sequence encoding a phosphoserine aminotransferase such as SerC. In one nonlimiting embodiment, the exogenous genetic molecule comprises SEQ ID NO: 2 or 7 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:2 or 7 or a functional fragment thereof. In one nonlimiting embodiment, the exogenous genetic molecule comprises a nucleic acid sequence encoding a racemase. In one nonlimiting embodiment, the racemase is an arginine/lysine racemase. In one nonlimiting embodiment, the exogenous genetic molecule comprises SEQ ID NO: 5 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:5 or a functional fragment thereof. Additional nonlimiting examples of exogenous genetic molecules include expression constructs of, for example, one or more enzymes which catalyze the biosynthesis of L-serine from 3-phosphoglycerate and synthetic operons of, for example, one or more enzymes which catalyze the biosynthesis of L-serine from 3-phosphoglycerate. In one nonlimiting embodiment, the expression construct or synthetic operon comprises a nucleic acid sequence encoding a D-3-phosphoglycerate dehydrogenase, a phosphoserine phosphatase and/or a phosphoserine aminotransferase. In one nonlimiting embodiment, the expression construct or synthetic operon comprises a nucleic acid sequence encoding a racemase such as, but not limited to, a lysine/arginine racemase.

In one nonlimiting embodiment, the organism is altered to express, overexpress, not express or express less of one or more molecules depicted in FIG. 1. In one nonlimiting embodiment, the molecule(s) comprise a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence corresponding to a molecule(s) depicted in FIG. 1, or a functional fragment thereof.

Yet another aspect of the present invention relates to means and processes for use of these means for biosynthesis of compounds involved in serine metabolism, including derivatives thereof and/or compounds related thereto.

### BRIEF DESCRIPTION OF THE FIGURES

FIG. 1 is a schematic depicting biosynthesis of compounds involved in serine metabolism.

FIGS. 2A and 2B show L-serine accumulation in examples of altered organisms of the present invention. FIG. 2A shows results from assays as described in Table 3 while FIG. 2B shows results from assays as described in Table 4 with glyA deleted.

### DETAILED DESCRIPTION

The present invention provides processes for the biosynthesis of compounds involved in serine metabolism, and/or derivatives thereof and/or compounds related thereto, as well as organisms altered to increase biosynthesis of compounds involved in serine metabolism, and/or derivatives thereof and/or compounds related thereto, genetic molecules, including exogenous genetic molecules, of these altered organisms, and bio-derived, bio-based, or fermentation-derived products biosynthesized or otherwise produced by any of these methods and/or altered organisms.

In one aspect of the present invention, an organism is engineered and/or redirected to produce compounds involved in serine metabolism, as well as derivatives and compounds related thereto, by alteration of the organism to overexpress one or more enzymes which catalyze the biosynthesis of L-serine from 3-phosphoglycerate. In one nonlimiting embodiment, the organism is altered to overexpress SerA, SerB and/or SerC. In one nonlimiting embodiment, the organism is altered to overexpress and polypeptide of EC 1.1.1.95, EC 2.6.1.52, and/or EC 3.1.3.3. Organisms produced in accordance with the present invention are useful in methods for biosynthesizing higher levels of compounds involved in serine metabolism, derivatives thereof, and compounds related thereto.

For purposes of the present invention, “compounds involved in serine metabolism” encompasses L-serine, and structurally related compounds thereof.

For purposes of the present invention, by “derivatives and compounds related thereto” it is meant to encompass compounds derived from the same substrates and/or enzymatic reactions as compounds involved in serine metabolism, byproducts of these enzymatic reactions and compounds with similar chemical structure including, but not limited to, structural analogs wherein one or more substituents of compounds involved in serine metabolism are replaced with alternative substituents. Examples of related compounds which could be produced include other C2 and C3 molecules with functional side-chains, such as ethanol, 2-(methylamino)-, ethanol, 2-methoxy, D-serine, 1,2,3-propanetriol, and ethanolamine.

For purposes of the present invention, by “higher levels of compounds involved in serine metabolism” it is meant that the altered organisms and methods of the present invention are capable of producing increased levels of compounds involved in serine metabolism and derivatives and compounds related thereto as compared to the same organism without alteration. For compounds containing carboxylic acid groups such as organic monoacids, hydroxyacids, amino acids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the process for biosynthesis of compounds involved in serine metabolism and derivatives and compounds related thereto of the present invention, an organism capable of producing compounds involved serine metabolism and derivatives and compounds related thereto is obtained. The organism is then altered to produce more compounds involved in serine metabolism and derivatives and compounds related thereto in the altered organism as compared to the unaltered organism.

In one nonlimiting embodiment, the organism is Cupriavidus necator (C. necator) or an organism with properties similar thereto. A nonlimiting embodiment of the organism is set for at lgcstandards-atcc with the extension.org/products/a11/17699.aspx?geo_country=gb#generalinformation of the world wide web.

C. necator (previously called Hydrogenomonas eutrophus, Alcaligenes eutropha, Ralstonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. C. necator does not naturally contain genes for Ser A/B/C and therefore does not express these enzymes. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Sillman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (LphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

For the process of the present invention, the organism is altered to overexpress one or more enzymes which catalyze the biosynthesis of L-serine from 3-phosphoglycerate.

In one nonlimiting embodiment, the organism is altered to overexpress a D-3-phosphoglycerate dehydrogenase or a polypeptide having the activity of a D-3-phosphoglycerate dehydrogenase. In one nonlimiting embodiment, the D-3-phosphoglycerate dehydrogenase is SerA. In one nonlimiting embodiment, the enzyme is SerA or SerA4 from C. glutamicum or C. necator, respectively. In one nonlimiting embodiment, the SerA comprises a polypeptide encoded by a nucleic acid sequence of SEQ ID NO: 1 or 6, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 1 or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 6 or a functional fragment thereof. In one nonlimiting embodiment, the D-3-phosphoglycerate dehydrogenase enzyme is classified in EC 1.1.1.95.

In one nonlimiting embodiment, the organism is altered to express a phosphoserine phosphatase or a polypeptide having the activity of a phosphoserine phosphatase. In one nonlimiting embodiment, the phosphoserine phosphatase is SerB. In one nonlimiting embodiment, the enzyme is SerB or SerB1 from E. coli or C. necator, respectively. In one nonlimiting embodiment, the SerB comprises a polypeptide encoded by a nucleic acid sequence of SEQ ID NO: 3, 4, 8 or 9, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 3, 4, 8 or 9 or a functional fragment thereof, or a polypeptide with similar enzymatic activities encoded by a nucleic acid sequence with at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO: 3, 4, 8 or 9 or a functional fragment thereof. In one nonlimiting embodiment, the phosphoserine phosphatase is classified in EC 3.1.3.3.

In one nonlimiting embodiment, the organism is altered to express a phosphoserine aminotransferase or a polypeptide having the activity of a phosphoserine aminotransferase. In one nonlimiting embodiment, the phosphoserine aminotransferase is SerC. In one nonlimiting embodiment, the SerC is from E. coli or C. necator. In one nonlimiting embodiment, the SerC comprises a polypeptide encoded by a nucleic acid sequence of SEQ ID NO: 2 or 7, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 2 or 7 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or 7 or a functional fragment thereof. In one nonlimiting embodiment, the phosphoserine aminotransferase is classified in EC 2.6.1.52.

In one nonlimiting embodiment, the nucleic acid sequence is codon optimized for C. necator.

In one nonlimiting embodiment, the organism is altered to express two or more of the enzymes of D-3-phosphoglycerate dehydrogenase, phosphoserine phosphatase and/or phosphoserine aminotransferase as disclosed herein.

In one nonlimiting embodiment, the organism is altered to express D-3-phosphoglycerate dehydrogenase, phosphoserine phosphatase and phosphoserine aminotransferase as disclosed herein.

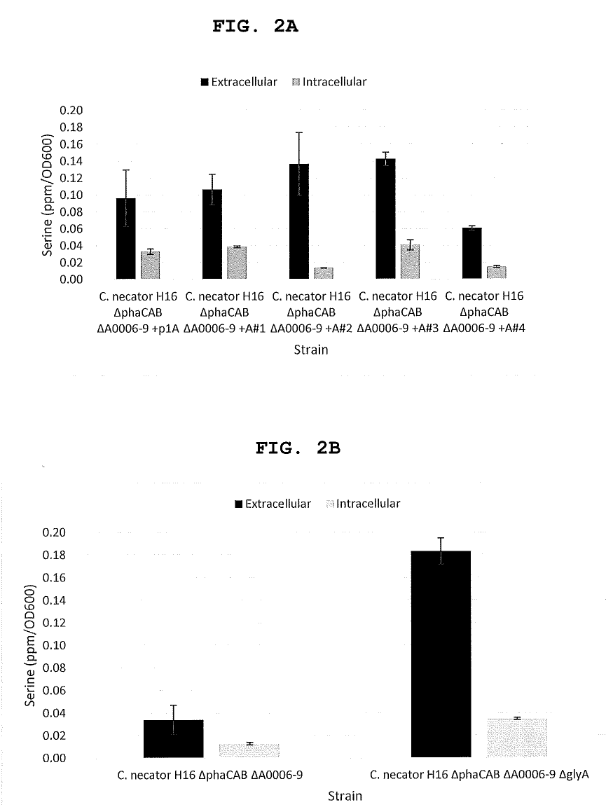

In one nonlimiting embodiment, the organism is further altered to eliminate one or more genes encoding enzymes utilizing L-serine by other pathways. Nonlimiting examples of such enzymes are set forth in Table 4. In one nonlimiting embodiment the eliminated one or more genes are sdaA and/or glyA (see FIG. 2B).

In one nonlimiting embodiment, the organism is further altered to comprise a racemase to divert the carbon flux from L-Serine to D-Serine. In one nonlimiting embodiment, the racemase is a lysine/arginine racemase. In one nonlimiting embodiment, the lysine/arginine racemase is argR from Pseudomonas taetrolens. In one nonlimiting embodiment, the racemase comprises a polypeptide encoded by a nucleic acid sequence of SEQ ID NO: 5, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 5 or a functional fragment thereof, or a polypeptide with similar enzymatic activities encoded by a nucleic acid sequence with at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO: 5 or a functional fragment thereof. In one nonlimiting embodiment, the racemase is classified in EC 5.1.1.5, EC 5.1.1.9 or EC 5.1.1.18.

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein compounds involved in serine metabolism and derivatives and compounds related thereto are produced.

In the process described herein, a fermentation strategy can be used that entails anaerobic, micro-aerobic or aerobic cultivation. A fermentation strategy can entail nutrient limitation such as nitrogen, phosphate or oxygen limitation.

Under conditions of nutrient limitation a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids. In Cupriavidus necator a modified form of overflow metabolism occurs in which excess carbon is sunk intracellularly into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31:168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

Applying a suitable nutrient limitation in defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, paper-pulp waste, black liquor, lignin, levulinic acid and formic acid, triglycerides, glycerol, fatty acids, agricultural waste, thin stillage, condensed distillers' solubles or municipal waste such as fruit peel/pulp. The non-biological feedstock can be, or can derive from, natural gas, syngas, CO₂/H₂, CO, H₂, O₂, methanol, ethanol, non-volatile residue (NVR), a caustic wash waste stream from cyclohexane oxidation processes or waste stream from a chemical industry such as, but not limited to a carbon black industry or a hydrogen-refining industry, or petrochemical industry, a nonlimiting example being a PTA-waste stream.

In one nonlimiting embodiment, at least one of the enzymatic conversions of the production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO₂/H₂, CO, H₂, O₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced compounds involved in serine metabolism or derivatives or compounds related thereto. Once produced, any method can be used to isolate the compound or compounds involved in serine metabolism or derivatives or compounds related thereto.

The present invention also provides altered organisms capable of biosynthesizing increased amounts of compounds involved in serine metabolism and derivatives and compounds related thereto as compared to the unaltered organism. In one nonlimiting embodiment, the altered organism of the present invention is a genetically engineered strain of Cupriavidus necator capable of producing compounds involved in serine metabolism and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing compounds involved in serine metabolism and derivatives and compounds related thereto via one or more enzymes which catalyze the biosynthesis of L-serine from 3-phosphoglycerate.

As used herein, a “substantially pure culture” of an altered organism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

In one nonlimiting embodiment, the altered organisms of the present invention comprise at least one genome-integrated synthetic and/or non-synthetic operon encoding an enzyme.

In one nonlimiting embodiment, the altered organism is produced by integration of a synthetic operon encoding one or more enzymes which catalyze the biosynthesis of L-serine from 3-phosphoglycerate. In one nonlimiting embodiment, the synthetic operon comprises a nucleic acid sequence encoding a D-3-phosphoglycerate dehydrogenase, a phosphoserine phosphatase and/or a phosphoserine aminotransferase.

In one nonlimiting embodiment, the D-3-phosphoglycerate dehydrogenase is SerA. In one nonlimiting embodiment, the enzyme is SerA or SerA4 from C. glutamicum or C. necator, respectively. In one nonlimiting embodiment, the SerA comprises a polypeptide encoded by a nucleic acid sequence of SEQ ID NO: 1 or 6, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 1 or 6 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 6 or a functional fragment thereof. In one nonlimiting embodiment, the D-3-phosphoglycerate dehydrogenase enzyme is classified in EC 1.1.1.95.

In one nonlimiting embodiment, the phosphoserine phosphatase is SerB. In one nonlimiting embodiment, the enzyme is SerB or SerB1 from E. coli or C. necator, respectively. In one nonlimiting embodiment, the SerB comprises a polypeptide encoded by a nucleic acid sequence of SEQ ID NO: 3, 4, 8 or 9, a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to a polypeptide encoded by a nucleic acid sequence set forth in SEQ ID NO: 3, 4, 8 or 9 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 3, 4, 8 or 9 or a functional fragment thereof. In one nonlimiting embodiment, the phosphoserine phosphatase is classified in EC 3.1.3.3.

In one nonlimiting embodiment, the phosphoserine aminotransferase is SerC. In one nonlimiting embodiment, the SerC is from E. coli or C. necator. In one nonlimiting embodiment, the SerC comprises a polypeptide encoded by a nucleic acid sequence of SEQ ID NO: 2 or 7, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 2 or 7 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 2 or 7 or a functional fragment thereof. In one nonlimiting embodiment, the phosphoserine aminotransferase is classified in EC 2.6.1.52.

In one nonlimiting embodiment, the nucleic acid sequence is codon optimized for C. necator.

In one nonlimiting embodiment, the organism is altered to express two or more of the enzymes of D-3-phosphoglycerate dehydrogenase, phosphoserine phosphatase and/or phosphoserine aminotransferase as disclosed herein.

In one nonlimiting embodiment, the organism is altered to express a D-3-phosphoglycerate dehydrogenase, a phosphoserine phosphatase and a phosphoserine aminotransferase as disclosed herein.

In one nonlimiting embodiment, the organism is further altered to eliminate one or more genes encoding enzymes utilizing L-serine by other pathways. In one nonlimiting embodiment the eliminated one or more genes are sdaA and/or glyA.

In one nonlimiting embodiment, the organism is further altered to comprise a racemase to divert the carbon flux from L-Serine to D-Serine. In one nonlimiting embodiment, the racemase is a lysine/arginine racemase. In one nonlimiting embodiment, the lysine/arginine racemase is argR from Pseudomonas taetrolens. In one nonlimiting embodiment, the racemase comprises a polypeptide encoded by a nucleic acid sequence of SEQ ID NO: 5, a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to a polypeptide encoded by a nucleic acid sequence set forth in SEQ ID NO: 5 or a functional fragment thereof, or a polypeptide with similar enzymatic activities encoded by a nucleic acid sequence with at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 5 or a functional fragment thereof. In one nonlimiting embodiment, the racemase is classified in EC 5.1.1.5, EC 5.1.1.9 or EC 5.1.1.18.

In one nonlimiting embodiment, the organism is further modified to eliminate phaCAB, involved in PHBs production and/or H16-A0006-9 encoding endonucleases thereby improving transformation efficiency.

The percent identity (and/or homology) between two amino acid sequences as disclosed herein can be determined as follows. First, the amino acid sequences are aligned using the BLAST 2 Sequences (B12seq) program from the stand-alone version of BLAST containing BLASTP version 2.0.14. This stand-alone version of BLAST can be obtained from the U.S. government's National Center for Biotechnology Information web site (www with the extension ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide fragment of a polypeptide or a nucleic acid sequence fragment encoding a peptide fragment of a polypeptide that has at least about 25% (e.g., at least about 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity.

Functional fragments may range in length from about 10% up to 99% (inclusive of all percentages in between) of the original sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine, hemagluttanin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates in the pathway through other enzymes acting on such intermediates. In one nonlimiting embodiment, the organism is further altered to eliminate one or more genes encoding enzymes utilizing L-serine by other pathways. In one nonlimiting embodiment the eliminated one or more genes are sdaA and/or glyA. In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids encoding a D-3-phosphoglycerate dehydrogenase, a phosphoserine phosphatase and a phosphoserine aminotransferase, and in some embodiments a racemase, as described herein, as well as modifications to endogenous genes.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host or organism once in or utilized by the host or organism. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence encoding one or more enzymes which catalyze the biosynthesis of L-serine from 3-phosphoglycerate. In one nonlimiting embodiment, the exogenous genetic molecule comprises a codon optimized nucleic acid sequence encoding a D-3-phosphoglycerate dehydrogenase, a phosphoserine phosphatase and/or a phosphoserine aminotransferase. In one nonlimiting embodiment, the nucleic acid sequence is codon optimized for C. necator.

In one nonlimiting embodiment, the exogenous genetic molecule comprises a nucleic acid sequence encoding the D-3-phosphoglycerate dehydrogenase SerA. In one nonlimiting embodiment, the nucleic acid sequence comprises SEQ ID NO:1 or 6 or a nucleic acid sequence encoding a polypeptide with similar enzymatic activities exhibiting at least about 50%, 60%, 70%, 75%, 80%, 85%, 90%, 910, 92%, 930, 94%, 95%, 96%, 97%, 98%, 99% or 99.5% sequence identity to the nucleic acid sequence set forth in SEQ ID NOs: 1 or 6 or a functional fragment thereof.

In one nonlimiting embodiment, the exogenous genetic molecule comprises a nucleic acid sequence encoding the phosphoserine phosphatase SerB. In one nonlimiting embodiment the nucleic acid sequence comprises SEQ ID NO:3, 4, 8 or 9 or a nucleic acid sequence encoding a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:3, 4 8 or 9 or a functional fragment thereof.

In one nonlimiting embodiment, the exogenous genetic molecule comprises a nucleic acid sequence encoding the phosphoserine aminotransferase SerC. In one nonlimiting embodiment, the exogenous genetic molecule comprises SEQ ID NO: 2 or 7 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:2 or 7 or a functional fragment thereof.

In one nonlimiting embodiment, the exogenous genetic molecule comprises a nucleic acid sequence encoding a racemase. In one nonlimiting embodiment, the exogenous genetic molecule comprises SEQ ID NO: 5 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:5 or a functional fragment thereof.

Additional nonlimiting examples of exogenous genetic molecules include expression constructs of, for example, a D-3-phosphoglycerate dehydrogenase, a phosphoserine phosphatase and/or a phosphoserine aminotransferase and synthetic operons of, for example, a D-3-phosphoglycerate dehydrogenase, a phosphoserine phosphatase and/or a phosphoserine aminotransferase. In some embodiments, the expression constructs and synthetic operons may comprise a nucleic acid sequence encoding for a racemase such as, but not limited to ArgR.

Also provided by the present invention are compounds involved in serine metabolism and derivatives and compounds related thereto bioderived from an altered organism according to any of methods described herein.

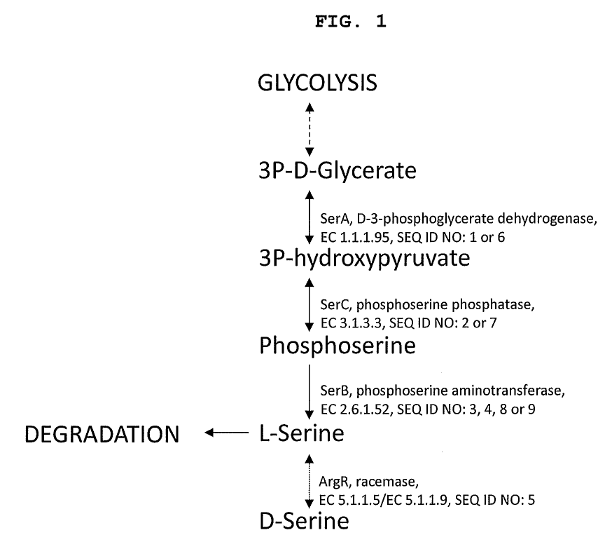

Further, the present invention relates to means and processes for use of these means for biosynthesis of compounds involved in serine metabolism and derivatives and compounds related thereto. Nonlimiting examples of such means include altered organisms and exogenous genetic molecules as described herein as well as any of the molecules as depicted in FIG. 1.

In addition, the present invention provides bio-derived, bio-based, or fermentation-derived products produced using the methods and/or altered organisms disclosed herein. In one nonlimiting embodiment, a bio-derived, bio-based or fermentation derived product is produced in accordance with the exemplary central metabolism depicted in FIG. 1. Examples of such products include, but are not limited to, compositions comprising at least one bio-derived, bio-based, or fermentation-derived compound or any combination thereof, as well as C2 and C3 compounds with functional side chains, molded substances, formulations and semi-solid or non-semi-solid streams comprising one or more of the bio-derived, bio-based, or fermentation-derived compounds or compositions, combinations or products thereof.

The following section provides further illustration of the methods and materials of the present invention. These Test Methods and Working Examples are illustrative only and are not intended to limit the scope of the invention in any way.

### Examples

### Selection of Genes for Overexpression

serA, serB, and serC encode enzymes catalyzing the biosynthesis of L-Serine from 3-phosphoglycerate, an intermediate of glycolysis. serA^{FR} from Corynebacterium glutamicum and serB and serC from E. coli were selected as well as C. necator genes serA4 and serB1 (see Table 2). C. necator H16 has 6 serA genes: A1-A6 (H16_A3712, H16_B0347, H16_B0466, H16_B0824, H16_B0841, H16_B1819). serA4 is closely related to serA of Corynebacterium glutamicum. C. necator H16 has 3 serB genes: H16_A1452 (serB1), H16_A3080 (serB2), and H16_B1164 (serB3). serB1 is the most similar to E. coli serB. See Table 1.

TABLE 1

| Gene | Full name | Source organism | Acc code | Justification |
|---|---|---|---|---|
| serA^{FR} | D-3- | Corynebacterium | A0A0U4XPS9 | Successful in |
| | phosphoglycerate | glutamicum | | E. coli: |
| | dehydrogenase | | | Gu et al |
| serB | Phosphoserine | E. coli | P0AGB0 | (2014) |
| | phosphatase | | | 41: 1443 |
| serC | Phosphoserine | E. coli | P23721 | |
| | aminotransferase | | | |
| serA4 | D-3- | C. necator | Q0K308 | Native for C. |
| H16_B0824 | phosphoglycerate | | | necator |
| | dehydrogenase | | | SerABC genes |
| serB1 | Phosphoserine | C. necator | Q0KBN4 | but status |
| H16_A1452 | phosphatase | | | “protein |
| serC | Phosphoserine | C. necator | Q0KDI3 | inferred from |
| H16_A0791 | aminotransferase | | | homology”. |
| argR | Lysine/arginine | Pseudomonas | I0J1I6 | Successful in: |
| | racemase | taetrolens | | Matsui et al |
| | | | | (2009) |
| | | | | 83: 1045 |

As the enzymes encoded by the E. coli or C. glutamicum serA gene are inhibited by their reaction product, a “FR”(=feedback resistant) version of the C. glutamicum was used which is a deletion mutant lacking four C-terminal residues of the wt protein. The corresponding serA genes from C. necator do not possess the domain required for the feedback inhibition and so the wt version of serA4 was used.

As accumulation of L-serine can be difficult due to the fact that L-serine is utilized to synthesize several other amino acids, nucleic acids and phospholipids, in some embodiment, a sink was used to trap L-serine passing through the node by turning L-serine into D-serine with a racemase, in particular lysine/arginine racemase, argR, from Pseudomonas taetrolens.

Four constructs were designed, as shown in Table 2, with two sets of biosynthetic genes, one being exogenous, another native for the organism. Each of the constructs was assembled using standard cloning techniques such as described, for example in Green and Sambrook, Molecular Cloning, A Laboratory Manual, Nov. 18, 2014 in two versions: with or without the abovementioned racemase.

TABLE 2

| Genes used in the assembly | SEQ ID NOs |
|---|---|
| cgSerA^{FR}-ecSerC-ecSerB | 1 + 2 + 3 |
| cgSerA^{FR}-ecSerC-ecSerB-ArgR | 1 + 2 + 3 + 5 |
| cnSerA4-cnSerC-cnSerB1 | 6 + 7 + 8 |
| cnSerA4-cnSerC-cnSerB1-ArgR | 6 + 7 + 8 + 5 |

### Selection of Genes for Deletion

KEGG metabolic pathways maps for C. necator H16 were analyzed to identify enzymes which utilize L-serine as a substrate. Table 3 provides list of these enzymes. Column DDF refers to expression levels of RNA as the end product formed when serine is used as a substrate. Deletions were made in these pathways so that any accumulating serine would not be used up as a substrate in pathways leading to pyruvate and other end products.

TABLE 3

| Leading to | EC | gene | | H16 | DDF |
|---|---|---|---|---|---|
| Pyruvate | 4.3.1.17 | sdaA | L-serine hydratase | H16_A3622 | 10.80 |
| | | — | L-serine hydratase, | H16_B0620 | 0.30 |
| | | | putative | | |
| | 4.3.1.19 | — | Threonine dehydratase | H16_A0427 | 17.09 |
| | | tdcB | Threonine deaminase | H16_B0554 | 6.34 |
| Tryptophane | 4.2.1.20 | trpA | tryptophan synthase | H16_A2612 | 24.64 |
| | | | alpha chain | | |
| | | trpB | tryptophan synthase beta | H16_A2614 | 41.09 |
| | | | chain | | |
| Glycine | 2.1.2.1 | glyA | glycine | H16_A2834 | 249.59 |
| | | | hydroxymethyltransferase | | |
| | 2.6.1.45 | — | aminotransferase | H16_B1170 | 3.19 |
| Q- | 2.7.8.8 | pssA | phosphatidyl synthase | H16_A1039 | 13.69 |
| phosphatidyl- | | — | phosphatidyl synthase | H16_B1122 | 9.10 |
| L-serine | | | | | |
| Q-acetyl-L- | 2.3.1.30 | cysE | serine Q- | H16_A1216 | 9.56 |
| serine | | | acetyltransferase | | |

Two gene deletions, one enzyme transforming L-serine to glycine (giyA) and another transforming L-serine to pyruvate (sdaA) were selected for the constructs.

### Cloning

The assemblies of genes intended for overexpression were designed as described in Table 1. Each gene was synthesized separately using standard cloning techniques. Sequences of the genes were codon optimized for expression in C. necator. Full sequences are depicted in the Sequence Listing. Assemblies were cloned into 1A vector (a derivative of pBBR1-MCS2 disclosed at sciencedirect with the extension.com/science/article/pii/0378111995005841 of the world wide web modified for compatability with the DNA assembly technique) and transformed first into E. coli DH5α (New England Biolabs). Colony PCR was used to screen colonies harboring the plasmid with insert of correct size. Initial DNA sequencing was performed with purified PCR products. This was followed by full assembly sequencing according to the method as described at eurofinsgenomics with the extension.eu/en/eurofins-genomics/product-faqs/custom-dna-sequencing/of the world wide web. Plasmids with confirmed sequence were transformed into a derivative of C. necator H16 ΔphaCAB ΔA0006-9 as described in U.S. patent application Ser. No. 15/717,216, teachings of which are incorporated herein by reference.

### Bioassay

For each strain to be tested, inoculums (10 ml) were prepared in TSB with respective antibiotic using standard procedures. Cells were subsequently washed in a defined minimal media before inoculation. After growth upon the defined minimal media with fructose as the carbon source, cells were induced with L-Arabinose.

Samples were then purified from cells and proteins by subsequent (i) centrifugation and (ii) filtration.

LC-MS was performed using an Agilent Technologies (Santa Clara, Calif., USA) 1290 Series Infinity HPLC system, coupled to an Agilent 6530 Series Q-TOF mass spectrometer. Manufacturer instructions were followed using a BEH Amide UPLC column: 2.1 mm diameter×50 mm length×1.7 μm particle size (Waters, Milford, Mass., USA).

External standard curves were used for quantitation. Calibration levels of 0.01, 0.02, 0.05, 0.1, 0.2, 0.5, 1, 2, 5 and 10 μg/ml were constructed in a matrix-matched solution, typically the blank medium, diluted to the same level as the samples in acetonitrile. Concentrations were determined by interpolation of sample responses against the calibration curve, using Agilent MassHunter Quantitative Analysis software.

TABLE 4
Sequence Information for Sequences in Sequence Listing

| SEQ ID | |
|---|---|
| NO: | Sequence Description |
| 1 | cgSerA^{FR} |
| 2 | ecSerC |
| 3 | ecSerB (without ArgR) |
| 4 | ecSerB (with ArgR) |
| 5 | ArgR |
| 6 | cnSerA4 |
| 7 | cnSerC |
| 8 | cnSerB1 (without ArgR) |
| 9 | cnSerB1 |

1. A process for the biosynthesis of compounds involved in serine metabolism, and/or derivatives thereof and/or compounds related thereto, said process comprising:
obtaining an organism capable of producing compounds involved in serine metabolism, and/or derivatives thereof and/or compounds related thereto;
altering the organism; and
producing more compounds involved in serine metabolism, and/or derivatives thereof and/or compounds related thereto by the altered organism as compared to the unaltered organism.

2. The process of claim 1 wherein the organism is C. necator or an organism with properties similar thereto.

3. The process of claim 1 wherein the organism is altered to overexpress one or more enzymes which catalyze the biosynthesis of L-serine from 3-phosphoglycerate.

4. The process of claim 3 wherein the organism is altered to overexpress a D-3-phosphoglycerate dehydrogenase, a phosphoserine phosphatase and/or a phosphoserine aminotransferase.

5-6. (canceled)

7. The process of claim 4 wherein the D-3-phosphoglycerate dehydrogenase is SerA, the phosphoserine phosphatase is SerB and/or the phosphoserine aminotransferase is SerC.

8. The process of claim 7 wherein the SerA is from C. glutamicum or C. necator, the SerB is from E. coli or C. necator and/or the SerC is from E. coli or C. necator.

9. The process of claim 4 wherein the D-3-phosphoglycerate dehydrogenase comprises a polypeptide encoded by a nucleic acid sequence of SEQ ID NO: 1 or 6, a polypeptide with similar enzymatic activities exhibiting at least about 50% sequence identity to a polypeptide encoded by a nucleic acid sequence set forth in SEQ ID NO: 1 or 6 or a functional fragment thereof, or a polypeptide with similar enzymatic activities encoded by a nucleic acid sequence with at least 50% sequence identity to the nucleic acid sequence set forth in SEQ ID NO: 1 or 6 or a functional fragment thereof.

10-11. (canceled)

12. The process of claim 4 wherein the phosphoserine phosphatase comprises a polypeptide encoded by a nucleic acid sequence of SEQ ID NO: 3, 4, 8 or 9, a polypeptide with similar enzymatic activities exhibiting at least about 50% sequence identity to a polypeptide encoded by a nucleic acid sequence set forth in SEQ ID NO: 3, 4, 8 or 9 or a functional fragment thereof, or a polypeptide with similar enzymatic activities encoded by a nucleic acid sequence with at least 50% sequence identity to the nucleic acid sequence set forth in SEQ ID NO: 3, 4, 8 or 9 or a functional fragment thereof.

13-14. (canceled)

15. The process of claim 4 wherein the phosphoserine aminotransferase comprises a polypeptide encoded by a nucleic acid sequence of SEQ ID NO: 2 or 7, a polypeptide with similar enzymatic activities exhibiting at least about 50% sequence identity to a polypeptide encoded by a nucleic acid sequence set forth in SEQ ID NO: 2 or 7 or a functional fragment thereof, or a polypeptide with similar enzymatic activities encoded by a nucleic acid sequence with at least 50% sequence identity to the nucleic acid sequence set forth in SEQ ID NO: 2 or 7 or a functional fragment thereof.

16. The process of claim 1 wherein the organism is further altered to eliminate one or more genes encoding enzymes utilizing L-serine by other pathways.

17. The process of claim 16 wherein the eliminated one or more genes are sdaA and/or glyA.

18. The process of claim 1 wherein the organism is further altered to comprise a racemase to divert the carbon flux from L-serine to D-serine.

19. The process of claim 18 wherein the racemase is a lysine/arginase racemase.

20. The process of claim 18 wherein the racemase is argR from Pseudomonas taetrolens.

21. The process of claim 18 wherein the racemase comprises a polypeptide encoded by a nucleic acid sequence of SEQ ID NO: 5, a polypeptide with similar enzymatic activities exhibiting at least about 50% sequence identity to a polypeptide encoded by a nucleic acid sequence set forth in SEQ ID NO: 5 or a functional fragment thereof, or a polypeptide with similar enzymatic activities encoded by a nucleic acid sequence with at least about 50% sequence identity to the nucleic acid sequence set forth in SEQ ID NO: 5 or a functional fragment thereof.

22. The process of claim 1 wherein the organism is further altered to eliminate phaCAB, involved in PHBs production and/or H16-A0006-9 encoding endonucleases thereby improving transformation efficiency.

23. (canceled)

24. An altered organism capable of producing more compounds involved in serine metabolism, derivatives thereof and/or compounds related thereto as compared to an unaltered organism.

25. The altered organism of claim 24 which is C. necator or an organism with properties similar thereto.

26. The altered organism of claim 24 which overexpresses one or more enzymes which catalyze the biosynthesis of L-serine from 3-phosphoglycerate.

27. The altered organism of claim 24 which overexpresses a D-3-phosphoglycerate dehydrogenase, a phosphoserine phosphatase and/or a phosphoserine aminotransferase.

28-29. (canceled)

30. The altered organism of claim 27 wherein the D-3-phosphoglycerate dehydrogenase is SerA, the phosphoserine phosphatase is SerB and/or the phosphoserine aminotransferase is SerC.

31. The altered organism of claim 30 wherein the SerA is from C. glutamicum or C. necator, the SerB is from E. coli or C. necator and/or the SerC is from E. coli or C. necator.

32. The altered organism of claim 27 wherein the D-3-phosphoglycerate dehydrogenase comprises a polypeptide encoded by a nucleic acid sequence of SEQ ID NO: 1 or 6, a polypeptide with similar enzymatic activities exhibiting at least about 50% sequence identity to a polypeptide encoded by a nucleic acid sequence set forth in SEQ ID NO: 1 or 6 or a functional fragment thereof, or a polypeptide with similar enzymatic activities encoded by a nucleic acid sequence with at least about 50% sequence identity to the nucleic acid sequence set forth in SEQ ID NO: 1 or 6 or a functional fragment thereof.

33-34. (canceled)

35. The altered organism of claim 27 wherein the phosphoserine phosphatase comprises a polypeptide encoded by a nucleic acid sequence of SEQ ID NO: 3, 4, 8 or 9, a polypeptide with similar enzymatic activities exhibiting at least about 50% sequence identity to a polypeptide encoded by a nucleic acid sequence set forth in SEQ ID NO: 3, 4, 8 or 9 or a functional fragment thereof, or a polypeptide with similar enzymatic activities encoded by a nucleic acid sequence with at least about 50% sequence identity to the nucleic acid sequence set forth in SEQ ID NO: 3, 4, 8 or 9 or a functional fragment thereof.

36-37. (canceled)

38. The altered organism of claim 27 wherein the phosphoserine aminotransferase comprises a polypeptide encoded by a nucleic acid sequence of SEQ ID NO: 2 or 7, a polypeptide with similar enzymatic activities exhibiting at least about 50% sequence identity to a polypeptide encoded by a nucleic acid sequence set forth in SEQ ID NO: 2 or 7 or a functional fragment thereof, or a polypeptide with similar enzymatic activities encoded by a nucleic acid sequence with at least about 50% sequence identity to the nucleic acid sequence set forth in SEQ ID NO: 2 or 7 or a functional fragment thereof.

39. The altered organism of claim 27 further altered to eliminate one or more genes encoding enzymes utilizing L-serine by other pathways.

40. The altered organism of claim 39 wherein the eliminated one or more genes are sdaA and/or glyA.

41. The altered organism of claim 24 wherein the organism is further altered to comprise a racemase to divert the carbon flux from L-Serine to D-Serine.

42. The altered organism of claim 41 wherein the racemase is a lysine/arginine racemase.

43. The altered organism of claim 41 wherein the racemase is argR from Pseudomonas taetrolens.

44. The altered organism of claim 41 wherein the racemase comprises a polypeptide encoded by a nucleic acid sequence of SEQ ID NO: 5, a polypeptide with similar enzymatic activities exhibiting at least about 50% sequence identity to a polypeptide encoded by a nucleic acid sequence set forth in SEQ ID NO: 5 or a functional fragment thereof, or a polypeptide with similar enzymatic activities encoded by a nucleic acid sequence with at least about 50% sequence identity to the nucleic acid sequence set forth in SEQ ID NO: 5 or a functional fragment thereof.

45. The altered organism of claim 24 wherein the organism is further altered to eliminate phaCAB, involved in PHBs production and/or H16-A0006-9 encoding endonucleases thereby improving transformation efficiency.

46. (canceled)

47. A bio-derived, bio-based, or fermentation-derived product produced from the method of claim 1, wherein said product comprises:
(i) a composition comprising at least one bio-derived, bio-based, or fermentation-derived compound or any combination thereof;
(ii) a bio-derived, bio-based, or fermentation-derived C2 and C3 compounds with functional side chains comprising the bio-derived, bio-based, or fermentation-derived composition or compound of (i), or any combination thereof;
(iii) a molded substance obtained by molding the bio-derived, bio-based, or fermentation-derived composition or compound of (i) or the bio-derived, bio-based, or fermentation-derived C2 and C3 compounds with functional side chains of (ii), or any combination thereof;
(iv) a bio-derived, bio-based, or fermentation-derived formulation comprising the bio-derived, bio-based, or fermentation-derived composition or compound of (i), the bio-derived, bio-based, or fermentation-derived C2 and C3 compounds with functional side chains of (ii), or the bio-derived, bio-based, or fermentation-derived molded substance of (iii), or any combination thereof; or
(v) a bio-derived, bio-based, or fermentation-derived semi-solid or a non-semi-solid stream, comprising the bio-derived, bio-based, or fermentation-derived composition or compound of (i), the bio-derived, bio-based, or fermentation-derived C2 and C3 compounds with functional side chains of (ii), the bio-derived, bio-based, or fermentation-derived formulation of (iii), or the bio-derived, bio-based, or fermentation-derived molded substance of (iv), or any combination thereof.

48. A bio-derived, bio-based or fermentation derived product produced in accordance with the central metabolism depicted in FIG. 1.

49. An exogenous genetic molecule of the altered organism of claim 24.

50. The exogenous genetic molecule of claim 49 comprising a codon optimized nucleic acid sequence or an expression construct or synthetic operon for one or more enzymes which catalyze the biosynthesis of L-serine from 3-phosphoglycerate, an expression construct or synthetic operon comprising a nucleic acid sequence encoding a D-3-phosphoglycerate dehydrogenase, a phosphoserine phosphatase and/or a phosphoserine aminotransferase, an expression construct or synthetic operon comprising a nucleic acid sequence encoding a racemase or an expression construct or synthetic operon comprising a nucleic acid sequence encoding ArgR.

51. The exogenous genetic molecule of claim 50 codon optimized for C. necator.

52. The exogenous genetic molecule of claim 49 comprising a codon optimized nucleic acid sequence encoding one or more enzymes which catalyze the biosynthesis of L-serine from 3-phosphoglycerate.

53. The exogenous genetic molecule of claim 49 comprising a codon optimized nucleic acid sequence encoding a D-3-phosphoglycerate dehydrogenase, a phosphoserine phosphatase and/or a phosphoserine aminotransferase.

54. The exogenous genetic molecule of claim 49 comprising SEQ ID NO:1 or 6 or a nucleic acid sequence encoding a polypeptide with similar enzymatic activities exhibiting at least about 50% sequence identity to the nucleic acid sequence set forth in SEQ ID NOs: 1 or 6 or a functional fragment thereof.

55. The exogenous genetic molecule of claim 49 comprising SEQ ID NO: 3, 4, 8 or 9 or a nucleic acid sequence encoding a polypeptide with similar enzymatic activities exhibiting at least about 50% sequence identity to the nucleic acid sequence set forth in SEQ ID NO: 3, 4, 8 or 9 or a functional fragment thereof.

56. The exogenous genetic molecule of claim 49 comprising SEQ ID NO: 2 or 7 or a nucleic acid sequence encoding a polypeptide with similar enzymatic activities exhibiting at least about 50% sequence identity to the nucleic acid sequence set forth in SEQ ID NO: 2 or 7 or a functional fragment thereof.

57. The exogenous genetic molecule of claim 49 comprising a nucleic acid sequence encoding a racemase.

58. The exogenous genetic molecule of claim 49 comprising SEQ ID NO: 5 or a nucleic acid sequence encoding a polypeptide with similar enzymatic activities exhibiting at least about 50% sequence identity to the nucleic acid sequence set forth in SEQ ID NO:5 or a functional fragment thereof.

59.-62. (canceled)

63. A process for the biosynthesis of compounds involved in serine metabolism, derivatives thereof and/or compounds related thereto, said process comprising providing a means capable of producing compounds involved in serine metabolism, derivatives thereof and/or compounds related thereto and producing compounds involved in serine metabolism, derivatives thereof and/or compounds related thereto with said means.

64. A process for biosynthesis of compounds involved in serine metabolism, and derivatives thereof, and compounds related thereto, said process comprising:
a step for performing a function of altering an organism capable of producing compounds involved in serine metabolism, derivatives thereof, and/or compounds related thereto such that the altered organism produces more compounds involved in serine metabolism, derivatives thereof, and/or compounds compared to a corresponding unaltered organism; and
a step for performing a function of producing compounds involved in serine metabolism, derivatives thereof, and/or compounds related thereto in the altered organism.

65-66. (canceled)


SEQUENCE LISTING (9 sequences)

SEQ ID NO: 1 - DNA, 1581 bp, Artificial sequence
  Synthetic
atgtcccaaaatggccgccccgtagttctgatcgccgacaagctcgcccagtccaccgtggacgccctgggtgatgcggtcgaagtccggtgggtcgacggcccaaaccgcccggaactgctggacgccgtgaaggaagccgacgccctcctggtgcgttcggccaccaccgtcgacgcagaagtgatcgcggcggcgccgaacctcaagatcgtcggtcgcgccggcgtcggcctggacaatgtggacatcccggcggccaccgaggcgggcgtgatggtggccaacgccccgacctcgaatatccactcggcctgcgagcacgccatctcgctgctgctcagcaccgcccgccagatcccagcggcagacgccacgctgcgcgagggcgagtggaagcgctcctcgttcaatggcgtggaaatcttcggcaagacagtgggcatcgtcggcttcggccacatcggccagctgtttgcacagcgcctggccgcattcgagactaccatcgtcgcctacgacccgtacgcgaacccggcgcgcgcggcgcaactgaacgtcgaactggtcgagctggacgaactcatgtcgcgcagcgacttcgtgactatccatttgccgaaaacgaaagaaactgccggcatgttcgacgcgcagctgttggcgaagtcgaagaaaggccagatcattatcaacgcggcgcgtggcggcctggtggatgaacaagccctcgcagacgcgattgagagcggccatatccggggtgccggcttcgatgtctatagcaccgagccttgcacggattcgcccctcttcaagctgccccaggtcgtggtgacccctcatctgggcgcctcgacggaagaagcacaggaccgcgcggggaccgacgtggccgacagcgtgctcaaggcgctcgccggcgagttcgtcgcagacgccgtgaacgtgtcggggggccgcgtgggtgaagaagtggcgctctggatggacttggcccgcaagctgggcctgctggccggtaagctggtcgatgccgcccccgtgtccatcgaagtcgaggcgcgcggcgaactgagctccgaacaagtggacgccctgggcctgtcggccgtccggggcctctttagcggcattatcgaagaatccgtcaccttcgtcaatgcaccgcggatcgcagaggagcgtgggctggacatcagcgtcaagacgaacagcgaaagcgtgacccaccgcagcgtgctccaagtgaaagtgattacgggctccggcgcgtcggcgaccgtcgtgggcgcgttgaccggcctcgaacgggtggagaagatcacccgcatcaacggccgcggcttggatctgcgtgcagagggcctcaatctctttctccagtacaccgacgccccgggggccctgggcacggtcgggaccaaactgggcgccgcgggcatcaacattgaggccgcggcgctcacccaggcagaaaagggcgatggcgccgtgctcatcctgcgcgtggaatcggccgtcagcgaggaactggaggccgagatcaatgccgagctcggcgccacgtccttccagtga

SEQ ID NO: 2 - DNA, 1089 bp, Artificial sequence
  Synthetic
atggcacaaatcttcaatttctcgtccggtcctgcaatgctccccgccgaagtgctcaagcaagcgcagcaagagctgcgcgattggaatggtctgggcacgtcggtgatggaagtcagccaccggggcaaagaatttatccaggtcgccgaagaagcggagaaagacttccgcgatctcctgaatgtcccgtcgaactacaaggtcctgttttgccacggtggcggccgtggccagttcgccgccgtgccgctcaacatcctgggcgacaagacaaccgcggactacgtggacgcgggctattgggcggcatccgccatcaaggaagcgaagaaatattgcacccccaacgtgttcgacgccaaggtgaccgtcgacggcctgcgtgcggtgaagccgatgcgcgaatggcagctctccgacaacgccgcgtacatgcattactgcccgaacgaaacgatcgacggcatcgcgatcgatgaaacgccggacttcggcgccgacgtcgtcgtcgcagcggacttcagctcgacgatcctgtcccgcccaatcgatgtgagccgctacggcgtgatctacgccggcgcgcagaagaacatcggcccggcgggcctgaccatcgtgatcgtgcgcgaggacttgttgggcaaggcgaacattgcgtgcccctcgattctggactactcgatcctgaatgacaacggttcgatgttcaacaccccgcccaccttcgcgtggtacctgagcggcctggtctttaagtggctgaaggccaatggcggggtcgccgagatggacaagatcaaccagcagaaagccgagctgctgtacggcgtgatcgataacagcgatttctatcggaacgacgtggccaaggccaaccgctcgcggatgaacgtgccgttccagctggcggactcggccctggacaaactgttcctggaagagagcttcgcggccgggctgcatgccctgaaggggcaccgcgtcgtgggcggcatgcgcgcctccatctacaatgcgatgccgctggaaggcgtcaaggccctcaccgatttcatggtcgagttcgagcgccgccacggctga

SEQ ID NO: 3 - DNA, 969 bp, Artificial sequence
  Synthetic
atgcccaatattacctggtgtgacttgcccgaagatgtgtcgttatggcctggcttgcccctgagcctgtccggcgacgaagtgatgcccctggactaccacgcgggccggtcgggctggctgctgtacgggcgcggtctggacaagcagcgcctgacccagtatcagtcgaagctgggcgccgcgatggtgatcgtggcggcgtggtgcgtcgaggactaccaagtgatccgcctggcgggcagcctgacggcgcgtgccacacgcctcgcacacgaagcccagctggatgtcgcgccgctgggcaagattccgcatctgcgtacgccgggcctgctggtgatggacatggactccacggccatccagatcgagtgcatcgatgagatcgccaagctcgccggtaccggcgaaatggtcgccgaagtcaccgagcgcgcgatgcggggcgaattggatttcacggcctccctgcgctcgcgcgtcgcaaccctgaagggcgcggacgcgaatatcctgcaacaggtccgcgaaaacctcccgctgatgccgggcctcacccagctcgtgttgaagctcgaaacgctgggctggaaagtcgcgatcgccagcggtggcttcaccttcttcgcggagtacttgcgcgacaagctgcgcctgaccgccgtggtggccaacgagctcgaaatcatggacggcaagtttacgggcaacgtgatcggcgacatcgtggacgcccaatacaaggccaagaccctgacacggctggcccaggaatacgagatcccgctggcacagaccgtcgccatcggggacggcgcgaacgatctgccgatgatcaaagccgccggcctggggatcgcgtatcacgccaagcccaaggtcaacgagaaagccgaagtgactatccgccacgcagacctgatgggcgtgttctgcatcctgagcggctcgctcaaccaaaagtga

SEQ ID NO: 4 - DNA, 969 bp, Artificial sequence; identical to SEQ ID NO: 3
  Synthetic

SEQ ID NO: 5 - DNA, 1230 bp, Artificial sequence
  Synthetic
atgcccttctcccgtactcttctcgcactctccctcggcatggcactgctacaaaacccggcgttcgcggcgccaccgctgtcgatgaccgacggcgtggcccaggtcaatacgcaggactcgaacgcctgggtggaaatcaataaagccgcgttcgaacacaacatccgtacgctgcagacggcgctggcgggcaagtcgcaaatctgcgccgtcctcaaggccgacgcgtacggccacggcatcggcctgctgatgccgagcgtgatcgcgatgggcgtcccgtgcgtgggcgtggcgtccaacgaagaggcacgggtcgtccgcgagtcgggctttaagggccagctgatccgcgtgcggacggccgcgctgtccgagctggaagcggccctgccgtacaacatggaagaactggtggggaacttggacttcgccgtgaaggcatcgctcatcgcagaggaccacggccgcccgctggtcgtgcatttgggcctgaactcctcgggcatgagccgcaatggcgtcgacatgaccacggcacaggggcgccgggacgccgtggcgatcaccaaggtgccgaacctggaagtccgcgccattatgacgcacttcgccgtcgaggacgcagccgacgtgcgcgccggcctgaaagccttcaaccaacaggcgcaatggttgatgaacgtggcgcagctggaccgcagcaagatcacgctccacgcggccaactcgttcgcgaccctggaagtgcccgagagccacctggatatggtccgtcccggcggcgccctcttcggcgacaccgtcccgagccatacggaatacaagcgcgtgatgcagttcaagtcccacgtggcgtccgtcaacagctacccgaagggtaacaccgtgggctacggccgcacctataccctgggtcgggactcgcgcctggccaatatcaccgtcggctattcggatgggtatcgccgggccttcactaacaagggcatcgtgctgatcaacgggcaccgcgtccccgtcgtgggtaaggtcagcatgaacaccctcatggtcgacgtgaccgatgcccctgacgtgaaatcgggcgacgaagtggtgctgtttggccatcagggcaaggccgagattacacaggccgagatcgaggacattaacggcgccttgctcgcggatctgtacaccgtctggggtaactcgaacccgaagatcctgaaggaccagtga

SEQ ID NO: 6 - DNA, 1218 bp, Artificial sequence
  Synthetic
atgcgcaaaactaatgtcaccaccgcgatcttgctcgaaaacatccaccagtcggccaacgcgcgcctggcggaagccggcctccaagtggagcggcgcaccggtgccctggcgggcgccgagctgcgccaggtgttggcgagccacgacgtggtgggcatccgttccgccacacatctgcgtgcggacgacatccgctcggcccctgaactgctgagcatcggctgcttctgcatcgggacgagccaggtcgatctggaagccgccaccaccgccggcatcccggtgttcaatgcgccattctcgaacacgcgctcggtggccgaactggtcgtcagcgaagccgtcatgctgctgcgccgcattccagagaagaacactctcgcgcacgcgggcaagtgggcgaagggcgcggccggcgccttcgaggcacgcggcaagacgatcgccatcgtcggctacggcaacatcggcagccaggtcggcgtgctggcggaatcgatggggatgcgcgtcgtgtactatgatgtgctggcacgcctgtcgcatgggagcgcccgtgccgcgggctcgctggaagaagccgtgtcccaggcagaagtggtgacgctgcatgtgccggccaccccgcgcacgcggaacatgatcgacgcccgcatcctggccgcctgcaagcccggtgcaattctgatcaacgcgtcgcgcggcagcgtggtcgacatcgccgcgctcgcggcagccctgcgcgagaagcgcttgggtggggcggccatcgatgtgttcccggaagaacccaagaccaacaatgatccgtttgtctccgagctgcagaacatgccgaacgtgctgctgaccccgcacgtcggcggctccaccgaagaggcacaggaaaacatcgggaccgaagtggccgcgaagctggcccacttcctcgccacgggcggcaccattggcgcggtgaatttccccgaggtcgacccgggtcccctccacgccccggcccgcctgctgaacgtgcatggcaacgcccccggcgccctggcggcactcaacacgctgctggcccaggagggcgtgaacatcaccggccaacatctgcaaacccgcggccacacgggctacgtggtcacggacctggaccgcgtgccgtcggaccagctgatgcaagcgctgcgtacccacgcgggcttcgcccgctcccgcctgatcctgcggtga

SEQ ID NO: 7 - DNA, 1197 bp, Artificial sequence
  Synthetic
atggggaacacaggctcgcatttttcaatcccgcgcttaatgaatgacccgcaaaacccggcactcgccggcatgatgcagcgcgccctggcggaacgggtgtataacttctccccggggcccgccgccctgcctgcggaagtgctgcagcaagcggcagaagagatgctgtcgtggcacggtacgggcgtgtccgtcatggaaatgtcgcaccgctcgcgcgagttcgaaagcattcacaacgaagccatcgcggatctccgcgagctgctgcacatcccagccaacttcaaagtcctgttcttgcagggtggcgcgatcggcgaaaacgccatcgtgccgctgaatctcatgcggctgcgctccgcggagcagccgaaggccgacttcgtggtgacggggacttggtcggtcaagaccgagcaagaagcccggcgctacggcgcggtgaacatcgcggcgacctcggaagcggaaaagtttcaccgcattccggacatcgcggattggaagctcagcgacgacgcgggctacgtgcacctctgcaccaacgaaaccatcgtgggtgtcgagttccaagacatccccgacatcggccaggtcaagggcgaccgcgtggtcgtggcggacgcgtcgtcgcacatcctgtcgcgtccgatcgattggagccgggtccaggtcgtgtacggtggcgcccagaagaacatcggcccggcaggggtcacgattgtgatcgtgcgcgacgacctgatcggccacgcccacccgctctgccccagcgccttcaactggcgcctggtggcagagcataactcgatgtacaacaccccccccacgtacgccatctatatcgcgggcctcgtgttcaagtggctgaagcgccagggcggcgtcccggcaatcgagcagcgcaacatcgccaaggcctcggccctgtacaactatctggaccaatcggacttttaccgcaacgaaattcacccgagctgccgctcccgcatgaatgtccccttcttcctgggcgacgagagccgtaatgaagtgttcctgcagcaagcccgcgccaacggcctggtccagctgaagggccataagaccgtcggcggcatgcgcgccagcatctacaacgccatgcccctggaaggcgtcatggccttggtggacttcatgcgcgagttcgagcgtacctccgcctga

SEQ ID NO: 8 - DNA, 837 bp, Artificial sequence
  Synthetic
atgccactcattctccagtccaccgcccccttgtcccccgccgacctcgatacagcgcgcacgctggcccgcgcgtcggaactggtgctgcgctcggaaaccgtggccgcggcagaggaatgcgcaccgctcaccccggcgctgcgggacgccctggacgacttctgcggcccccgcgcgatcgactgggccgtcgtcccggccggtcgcaagttgagcgacttccacctggtcgcgatggatatggacagcacgctgatcacgatcgagtgcatcgatgaaatcgcggacttttgcgggctcaaagccgaggtgagcgccatcaccgaagcggcgatgcgcggcgagatcaccgatttcaacgaatcgctgcgccgtcgcgtggcgctgctgaagggcctggacgcctcggtgttggaccgggtgtacgaggaacgtctgcgcctctccccgggcgccgagcgcatgctccagaccgtgcaggccctgggcctgcgtactctgctggtgagcggcggcttcgtccacttcaccgagaaactgaaggcacggctgcagctcgatgtgacccgggcaaacacgctggaaatcgtcgacggcaagctgacgggcaacgtcgtgggggaaatcgtgaacgccgacgtcaaggcccggaccgtccaagaggtctgcgcgcgcattggtgccacgccggaccaggccatcgtgatgggcgacggcagcaatgacctgaagatgatggccgtggcgggcctgtcggtggcgttccgcgccaagcctgtggtccgcacccaggcgtcggtggccttcaaccacgtcggcctggacggcctgctg

SEQ ID NO: 9 - DNA, 855 bp, Artificial sequence
  Synthetic
atgccactcattctccagtccaccgcccccttgtcccccgccgacctcgatacagcgcgcacgctggcccgcgcgtcggaactggtgctgcgctcggaaaccgtggccgcggcagaggaatgcgcaccgctcaccccggcgctgcgggacgccctggacgacttctgcggcccccgcgcgatcgactgggccgtcgtcccggccggtcgcaagttgagcgacttccacctggtcgcgatggatatggacagcacgctgatcacgatcgagtgcatcgatgaaatcgcggacttttgcgggctcaaagccgaggtgagcgccatcaccgaagcggcgatgcgcggcgagatcaccgatttcaacgaatcgctgcgccgtcgcgtggcgctgctgaagggcctggacgcctcggtgttggaccgggtgtacgaggaacgtctgcgcctctccccgggcgccgagcgcatgctccagaccgtgcaggccctgggcctgcgtactctgctggtgagcggcggcttcgtccacttcaccgagaaactgaaggcacggctgcagctcgatgtgacccgggcaaacacgctggaaatcgtcgacggcaagctgacgggcaacgtcgtgggggaaatcgtgaacgccgacgtcaaggcccggaccgtccaagaggtctgcgcgcgcattggtgccacgccggaccaggccatcgtgatgggcgacggcagcaatgacctgaagatgatggccgtggcgggcctgtcggtggcgttccgcgccaagcctgtggtccgcacccaggcgtcggtggccttcaaccacgtcggcctggacggcctgctggcactgttcccgcattga